(19) United States
(12) Patent Application Publication
(10) Pub. No.: US 2019/0223748 A1
(43) Pub. Date: Jul. 25, 2019

(54) METHODS AND APPARATUS FOR MITIGATING NEUROMUSCULAR SIGNAL ARTIFACTS

(71) Applicant: CTRL-labs Corporation, New York, NY (US)

(72) Inventors: Adam Al-natsheh, New York, NY (US); Tudor Giurgica-Tiron, Stanford, CA (US); Qiushi Mao, Brooklyn, NY (US); Patrick Kaifosh, New York, NY (US)

(73) Assignee: CTRL-labs Corporation, New York, NY (US)

(21) Appl. No.: 16/258,279

(22) Filed: Jan. 25, 2019

Related U.S. Application Data
(60) Provisional application No. 62/621,829, filed on Jan. 25, 2018.

(51) Int. Cl. A61B 5/0488; A61B 5/00; A61B 5/04

(52) U.S. Cl. CPC A61B 5/0488; A61B 5/7264; A61B 5/04001; A61B 5/04012; A61B 5/7207

(57) ABSTRACT

Methods and apparatus for mitigating neuromuscular signal artifacts are described. The method comprises detecting in real-time, by at least one computer processor, one or more artifacts in a plurality of neuromuscular signals recorded by a plurality of neuromuscular sensors, determining, based at least in part, on the detected one or more artifacts, a plurality of derived neuromuscular signals to mitigate the one or more artifacts, and providing, as input to one or more trained statistical models, the plurality of derived neuromuscular signals.

### RELATED APPLICATIONS

This application claims priority under 35 U.S.C. § 119(e) to U.S. Provisional Application Ser. No. 62/621,829, entitled “METHODS AND APPARATUS FOR MITIGATING NEUROMUSCULAR SIGNAL ARTIFACTS,” filed on Jan. 25, 2018, which is incorporated by reference in its entirety.

### BACKGROUND

In some computer applications that generate representations of the human body, it is desirable for the application to know the spatial positioning, orientation and movement of a user's body to provide a realistic representation of body movement. For example, in a virtual reality (VR) environment, tracking the spatial position of the user's hand enables the application to represent hand motion in the VR environment, which allows the user to interact with (e.g., by grasping or manipulating) virtual objects within the VR environment. Some existing techniques for tracking movements of a user's body using wearable sensors include using information obtained from multiple Inertial Measurement Units (IMUs) affixed to different parts of the user's body, and using external imaging devices (e.g., fixed-position cameras) to reconstruct the position and orientation of parts of the user's body.

### SUMMARY

Some embodiments are directed to predicting information about the positioning and movements of portions of a user's arm and/or hand represented as a multi-segment articulated rigid body system with joints connecting the multiple segments of the rigid body system. Signals recorded by wearable neuromuscular sensors placed at locations on the user's body are provided as input to a statistical model trained to predict estimates of the position (e.g., absolute position, relative position, orientation) and forces associated with a plurality of rigid segments in a computer-based musculoskeletal representation associated with a hand when a user performs one or more movements. The combination of position information and force information associated with segments of a musculoskeletal representation associated with a hand is colloquially referred to herein as a “handstate” of the musculoskeletal representation. As a user performs different movements, a trained statistical model interprets neuromuscular signals recorded by the wearable neuromuscular sensors into position and force estimates (handstate information) that are used to update the musculoskeletal representation. As the neuromuscular signals are continuously recorded, the musculoskeletal representation is updated in real time (or near real-time) and a visual representation of a hand (e.g., within a virtual reality environment) is optionally rendered based on the current handstate estimates.

Other embodiments are directed to a computerized system. The computerized system comprises a plurality of neuromuscular sensors configured to continuously record a plurality of neuromuscular signals from a user, wherein the plurality of neuromuscular sensors are arranged on one or more wearable devices and at least one computer processor. The at least one computer processor is programmed to detect, in real-time (or near real-time), one or more artifacts in the recorded plurality of neuromuscular signals, determine, based at least in part, on the detected one or more artifacts, a plurality of derived neuromuscular signals to mitigate the one or more artifacts, and provide, as input to one or more trained statistical models, the plurality of derived neuromuscular signals.

In one aspect, detecting one or more artifacts in the recorded plurality of neuromuscular signals comprises determining, for each of the plurality of neuromuscular sensors at least one quality metric associated with the plurality of neuromuscular signals recorded by the neuromuscular sensor; and detecting one or more artifacts in the recorded plurality of neuromuscular signals based on the at least one quality metric.

In another aspect, detecting one or more artifacts in the recorded plurality of neuromuscular signals comprises identifying a first neuromuscular sensor in which the at least one quality metric deviates from a threshold value by more than a particular amount, and determining, based at least in part on the detected one or more artifacts, a plurality of derived neuromuscular signals comprises: processing the neuromuscular signals recorded by the first neuromuscular sensor.

In another aspect, processing the neuromuscular signals comprises filtering the neuromuscular signals recorded by the first neuromuscular sensor to remove at least one external noise component.

In another aspect, processing the neuromuscular signals comprises substituting the neuromuscular signals with previously recorded neuromuscular signals from the first neuromuscular sensor.

In another aspect, detecting one or more artifacts in the recorded plurality of neuromuscular signals comprises identifying a first neuromuscular sensor in which at least one quality metric deviates from a threshold value by more than a particular amount, and determining, based at least in part on the detected one or more artifacts, a plurality of derived neuromuscular signals comprises: replacing the neuromuscular signals recorded by the first neuromuscular sensor with neuromuscular signals recorded by at least one second neuromuscular sensor of the plurality of neuromuscular sensors.

In another aspect, replacing the neuromuscular signals recorded by the first neuromuscular sensor comprises replacing the neuromuscular signals with an average of neuromuscular signals recorded by two or more other neuromuscular sensors of the plurality of neuromuscular sensors.

In another aspect, the two or more other neuromuscular sensors are arranged adjacent to the first neuromuscular sensor on the one or more wearable devices.

In another aspect, detecting one or more artifacts in the recorded plurality of neuromuscular signals comprises identifying a first neuromuscular sensor in which at least one quality metric deviates from a threshold value by more than a particular amount, and determining, based at least in part on the detected one or more artifacts, a plurality of derived neuromuscular signals comprises: determining, based on the at least one quality metric, whether to process the neuromuscular signals recorded by the first neuromuscular sensor to at least partially remove the one or more artifacts or whether to replace the neuromuscular signals recorded by the first neuromuscular sensor with neuromuscular signals recorded by at least one second neuromuscular sensor of the plurality of neuromuscular sensors; processing the neuromuscular signals recorded by the first neuromuscular sensor when it is determined to process the neuromuscular signals recorded by the first neuromuscular sensor; and replacing the neuromuscular signals recorded by the first neuromuscular sensor with neuromuscular signals recorded by at least one second neuromuscular sensor of the plurality of neuromuscular sensors when it is determined to replace the neuromuscular signals recorded by the first neuromuscular sensor.

In another aspect, determining whether to process the neuromuscular signals recorded by the first neuromuscular sensor or replace the neuromuscular signals recorded by the first neuromuscular sensor is based, at least in part, on a type of artifact associated with neuromuscular signals recorded by the first neuromuscular sensor.

In another aspect, determining whether to process the neuromuscular signals recorded by the first neuromuscular sensor or replace the neuromuscular signals recorded by the first neuromuscular sensor is based, at least in part, on a magnitude of the at least one quality metric.

In another aspect, the one or more artifacts are selected from the group consisting of noise artifacts, skin-contact artifacts, skin lift-off artifacts, power line frequency (e.g., 50 Hz, 60 Hz) artifacts, clipped signal artifacts, inactive sensor artifacts, microfriction artifacts, and data degeneration artifacts.

In another aspect, detecting the one or more artifacts in the recorded plurality of neuromuscular signals comprises analyzing the plurality of neuromuscular signals with a plurality of detector circuits, wherein each of the detector circuits is configured to detect a particular artifact.

In another aspect, the one or more trained statistical models include at least one trained statistical model trained using neuromuscular signals including the one or more artifacts.

In another aspect, the at least one trained statistical model is trained using derived neuromuscular signals that mitigate, at least in part, the one or more artifacts.

In another aspect, the at least one computer processor is further programmed to generate a musculoskeletal representation of a portion of a user based, at least in part, on an output of the one or more trained statistical models.

In another aspect, the musculoskeletal representation of the portion of the user is a musculoskeletal representation of a hand of the user.

In another aspect, the musculoskeletal representation of the hand includes position information and force information determined based, at least in part, on the output of the one or more trained statistical models.

In another aspect, the plurality of neuromuscular sensors comprise electromyography (EMG) sensors, mechanomyography (MMG) sensors, sonomyography (SMG) sensors, or a combination of two or more of EMG, MMG and SMG sensors.

Other embodiments are directed to a method of mitigating neuromuscular signal artifacts. The method comprises detecting in real-time, by at least one computer processor, one or more artifacts in a plurality of neuromuscular signals recorded by a plurality of neuromuscular sensors, determining, based at least in part, on the detected one or more artifacts, a plurality of derived neuromuscular signals to mitigate the one or more artifacts; and providing, as input to one or more trained statistical models, the plurality of derived neuromuscular signals.

Other embodiments are directed to a computer-readable medium encoded with a plurality of instructions that, when executed by at least one computer processor perform a method. The method comprises detecting in real-time, one or more artifacts in a plurality of neuromuscular signals recorded by a plurality of neuromuscular sensors, determining, based at least in part, on the detected one or more artifacts, a plurality of derived neuromuscular signals to mitigate the one or more artifacts, and providing, as input to one or more trained statistical models, the plurality of derived neuromuscular signals.

Other embodiments are directed to a computerized system, comprising: a plurality of neuromuscular sensors configured to continuously record a plurality of neuromuscular signals from a user, wherein the plurality of neuromuscular sensors are arranged on one or more wearable devices; and at least one computer processor programmed to: determine, for each of the plurality of neuromuscular sensors at least one quality metric associated with the plurality of neuromuscular signals recorded by the neuromuscular sensor; detect, in real-time, one or more artifacts in the recorded plurality of neuromuscular signals based on the at least one quality metric, wherein detecting one or more artifacts in the recorded plurality of neuromuscular signals comprises identifying a first neuromuscular sensor in which at least one quality metric deviates from a threshold value by more than a particular amount; determine, based at least in part, on the detected one or more artifacts, a plurality of derived neuromuscular signals to mitigate the one or more artifacts, wherein determining the plurality of derived neuromuscular signals comprises: selecting, based on the at least one quality metric, an artifact mitigation technique to mitigate the detected one or more artifacts; and applying the artifact mitigation technique to the recorded plurality of neuromuscular signals to generate a plurality of derived neuromuscular signals in which the detected one or more artifacts have been at least partially removed; and provide, as input to one or more trained statistical models, the plurality of derived neuromuscular signals.

Other embodiments are directed to a method of mitigating neuromuscular signal artifacts, the method comprising: continuously recording a plurality of neuromuscular signals from a user using a plurality of neuromuscular sensors arranged on one or more wearable devices; determining, for each of the plurality of neuromuscular sensors at least one quality metric associated with the plurality of neuromuscular signals recorded by the neuromuscular sensor; detecting, in real-time, one or more artifacts in the recorded plurality of neuromuscular signals based on the at least one quality metric, wherein detecting one or more artifacts in the recorded plurality of neuromuscular signals comprises identifying a first neuromuscular sensor in which at least one quality metric deviates from a threshold value by more than a particular amount; determining, based at least in part, on the detected one or more artifacts, a plurality of derived neuromuscular signals to mitigate the one or more artifacts, wherein determining the plurality of derived neuromuscular signals comprises: selecting, based on the at least one quality metric, an artifact mitigation technique to mitigate the detected one or more artifacts; and applying the artifact mitigation technique to the recorded plurality of neuromuscular signals to generate a plurality of derived neuromuscular signals in which the detected one or more artifacts have been at least partially removed; and providing as input to one or more trained statistical models, the plurality of derived neuromuscular signals.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale.

FIG. 1 is a schematic diagram of a computer-based system for generating a musculoskeletal representation based on neuromuscular sensor data in accordance with some embodiments of the technology described herein;

FIG. 2 is flowchart of a process for mitigating neuromuscular signal artifacts in accordance with some embodiments of the technology described herein;

FIG. 3 is a flowchart of a process for mitigating neuromuscular signal artifacts using multiple detector circuits in accordance with some embodiments of the technology described herein;

FIG. 4 is a flowchart of a process for training a statistical model using training data determined based on neuromuscular signal data with simulated artifacts in accordance with some embodiments of the technology described herein;

FIG. 5 is a flowchart of an illustrative process for generating a statistical model for predicting musculoskeletal position information using signals recorded from sensors, in accordance with some embodiments of the technology described herein;

FIG. 6A illustrates a wearable system with sixteen EMG sensors arranged circumferentially around an elastic band configured to be worn around a user's lower arm or wrist, in accordance with some embodiments of the technology described herein;

FIG. 6B is a cross-sectional view through one of the sixteen EMG sensors illustrated in FIG. 6A; and

FIGS. 7A and 7B schematically illustrate components of a computer-based system on which some embodiments are implemented. FIG. 7A illustrates a wearable portion of the computer-based system and FIG. 7B illustrates a dongle portion connected to a computer, wherein the dongle portion is configured to communicate with the wearable portion.

### DETAILED DESCRIPTION

All or portions of the human musculoskeletal system can be modeled as a multi-segment articulated rigid body system, with joints forming the interfaces between the different segments and joint angles defining the spatial relationships between connected segments in the model. Constraints on the movement at the joints are governed by the type of joint connecting the segments and the biological structures (e.g., muscles, tendons, ligaments) that restrict the range of movement at the joint. For example, the shoulder joint connecting the upper arm to the torso and the hip joint connecting the upper leg to the torso are ball and socket joints that permit extension and flexion movements as well as rotational movements. By contrast, the elbow joint connecting the upper arm and the forearm and the knee joint connecting the upper leg and the lower leg allow for a more limited range of motion. As described herein, a multi-segment articulated rigid body system is used to model portions of the human musculoskeletal system. However, it should be appreciated that some segments of the human musculoskeletal system (e.g., the forearm), though approximated as a rigid body in the articulated rigid body system, may include multiple rigid structures (e.g., the ulna and radius bones of the forearm) that provide for more complex movement within the segment that is not explicitly considered by the rigid body model. Accordingly, a model of an articulated rigid body system for use with some embodiments of the technology described herein may include segments that represent a combination of body parts that are not strictly rigid bodies.

In kinematics, rigid bodies are objects that exhibit various attributes of motion (e.g., position, orientation, angular velocity, acceleration). Knowing the motion attributes of one segment of the rigid body enables the motion attributes for other segments of the rigid body to be determined based on constraints in how the segments are connected. For example, the hand may be modeled as a multi-segment articulated body with the joints in the wrist and each finger forming the interfaces between the multiple segments in the model. In some embodiments, movements of the segments in the rigid body model can be simulated as an articulated rigid body system in which position (e.g., actual position, relative position, or orientation) information of a segment relative to other segments in the model are predicted using a trained statistical model, as described in more detail below.

The portion of the human body approximated by a musculoskeletal representation as described herein as one non-limiting example, is a hand or a combination of a hand with one or more arm segments and the information used to describe a current state of the positional relationships between segments and force relationships for individual segments or combinations of segments in the musculoskeletal representation is referred to herein as the handstate of the musculoskeletal representation. It should be appreciated, however, that the techniques described herein are also applicable to musculoskeletal representations of portions of the body other than the hand including, but not limited to, an arm, a leg, a foot, a torso, a neck, or any combination of the foregoing.

In addition to spatial (e.g., position/orientation) information, some embodiments are configured to predict force information associated with one or more segments of the musculoskeletal representation. For example, linear forces or rotational (torque) forces exerted by one or more segments may be estimated. Examples of linear forces include, but are not limited to, the force of a finger or hand pressing on a solid object such as a table, and a force exerted when two segments (e.g., two fingers) are pinched together. Examples of rotational forces include, but are not limited to, rotational forces created when segments in the wrist or fingers are twisted or flexed. In some embodiments, the force information determined as a portion of a current handstate estimate includes one or more of pinching force information, grasping force information, or information about co-contraction forces between muscles represented by the musculoskeletal representation.

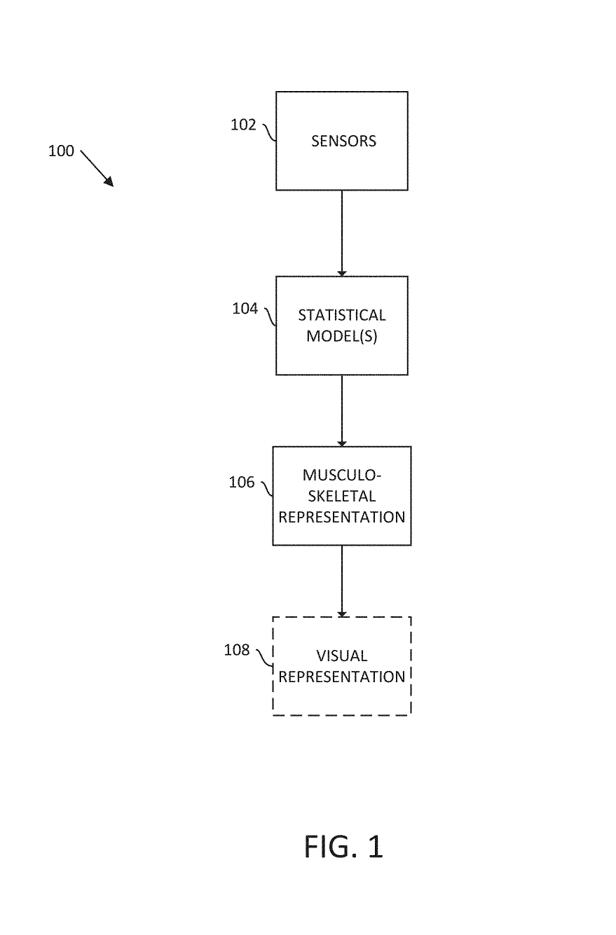

FIG. 1 illustrates a system 100 in accordance with some embodiments. The system includes a plurality of sensors 102 configured to record signals resulting from the movement of portions of a human body. Sensors 102 may include autonomous sensors. As used herein, the term “autonomous sensors” refers to sensors configured to measure the movement of body segments without requiring the use of external devices. In some embodiments, sensors 102 may also include non-autonomous sensors in combination with autonomous sensors. As used herein, the term “non-autonomous sensors” refers to sensors configured to measure the movement of body segments using external devices. Examples of external devices that include non-autonomous sensors include, but are not limited to, wearable (e.g. body-mounted) cameras, global positioning systems, and laser scanning systems.

Autonomous sensors may include a plurality of neuromuscular sensors configured to record signals arising from neuromuscular activity in skeletal muscle of a human body. The term “neuromuscular activity” as used herein refers to neural activation of spinal motor neurons that innervate a muscle, muscle activation, muscle contraction, or any combination of the neural activation, muscle activation, and muscle contraction. Neuromuscular sensors may include one or more electromyography (EMG) sensors, one or more mechanomyography (MMG) sensors, one or more sonomyography (SMG) sensors, a combination of two or more types of EMG sensors, MMG sensors, and SMG sensors, and/or one or more sensors of any suitable type that are configured to detect neuromuscular signals. In some embodiments, the plurality of neuromuscular sensors may be used to sense muscular activity related to a movement of the part of the body controlled by muscles from which the neuromuscular sensors are arranged to sense the muscle activity. Spatial information (e.g., position and/or orientation information) and force information describing the movement may be predicted based on the sensed neuromuscular signals as the user moves over time.

Autonomous sensors may include one or more Inertial Measurement Units (IMUs), which measure a combination of physical aspects of motion, using, for example, an accelerometer, a gyroscope, a magnetometer, or any combination of one or more accelerometers, gyroscopes and magnetometers. In some embodiments, IMUs may be used to sense information about the movement of the part of the body on which the IMU is attached and information derived from the sensed data (e.g., position and/or orientation information) may be tracked as the user moves over time. For example, one or more IMUs may be used to track movements of portions of a user's body proximal to the user's torso relative to the sensor (e.g., arms, legs) as the user moves over time.

In embodiments that include at least one IMU and a plurality of neuromuscular sensors, the IMU(s) and neuromuscular sensors may be arranged to detect movement of different parts of the human body. For example, the IMU(s) may be arranged to detect movements of one or more body segments proximal to the torso (e.g., an upper arm), whereas the neuromuscular sensors may be arranged to detect movements of one or more body segments distal to the torso (e.g., a forearm or wrist). It should be appreciated, however, that autonomous sensors may be arranged in any suitable way, and embodiments of the technology described herein are not limited based on the particular sensor arrangement. For example, in some embodiments, at least one IMU and a plurality of neuromuscular sensors may be co-located on a body segment to track movements of body segment using different types of measurements. In one implementation described in more detail below, an IMU sensor and a plurality of EMG sensors are arranged on a wearable device configured to be worn around the lower arm or wrist of a user. In such an arrangement, the IMU sensor may be configured to track movement information (e.g., positioning and/or orientation over time) associated with one or more arm segments, to determine, for example whether the user has raised or lowered their arm, whereas the EMG sensors may be configured to determine movement information associated with wrist or hand segments to determine, for example, whether the user has an open or closed hand configuration.

Each of the autonomous sensors includes one or more sensing components configured to sense information about a user. In the case of IMUs, the sensing components may include one or more accelerometers, gyroscopes, magnetometers, or any combination thereof to measure characteristics of body motion, examples of which include, but are not limited to, acceleration, angular velocity, and sensed magnetic field around the body. In the case of neuromuscular sensors, the sensing components may include, but are not limited to, electrodes configured to detect electric potentials on the surface of the body (e.g., for EMG sensors) vibration sensors configured to measure skin surface vibrations (e.g., for MMG sensors), and acoustic sensing components configured to measure ultrasound signals (e.g., for SMG sensors) arising from muscle activity.

In some embodiments, the output of one or more of the sensing components may be processed using hardware signal processing circuitry (e.g., to perform amplification, filtering, and/or rectification). In other embodiments, at least some signal processing of the output of the sensing components may be performed in software. Thus, signal processing of autonomous signals recorded by the autonomous sensors may be performed in hardware, software, or by any suitable combination of hardware and software, as aspects of the technology described herein are not limited in this respect.

In some embodiments, the recorded sensor data may be processed to compute additional derived measurements that are then provided as input to a statistical model, as described in more detail below. For example, recorded signals from an IMU sensor may be processed to derive an orientation signal that specifies the orientation of a rigid body segment over time. Autonomous sensors may implement signal processing using components integrated with the sensing components, or at least a portion of the signal processing may be performed by one or more components in communication with, but not directly integrated with the sensing components of the autonomous sensors.

In some embodiments, at least some of the plurality of autonomous sensors are arranged as a portion of a wearable device configured to be worn on or around part of a user's body. For example, in one non-limiting example, an IMU sensor and a plurality of neuromuscular sensors are arranged circumferentially around an adjustable and/or elastic band such as a wristband or armband configured to be worn around a user's wrist or arm. Alternatively, at least some of the autonomous sensors may be arranged on a wearable patch configured to be affixed to a portion of the user's body. In some embodiments, multiple wearable devices, each having one or more IMUs and/or neuromuscular sensors included thereon may be used to predict musculoskeletal position information for movements that involve multiple parts of the body.

In some embodiments, sensors 102 only includes a plurality of neuromuscular sensors (e.g., EMG sensors). In other embodiments, sensors 102 includes a plurality of neuromuscular sensors and at least one “auxiliary” sensor configured to continuously record a plurality of auxiliary signals. Examples of auxiliary sensors include, but are not limited to, other autonomous sensors such as IMU sensors, and non-autonomous sensors such as an imaging device (e.g., a camera), a radiation-based sensor for use with a radiation-generation device (e.g., a laser-scanning device), or other types of sensors such as a heart-rate monitor.

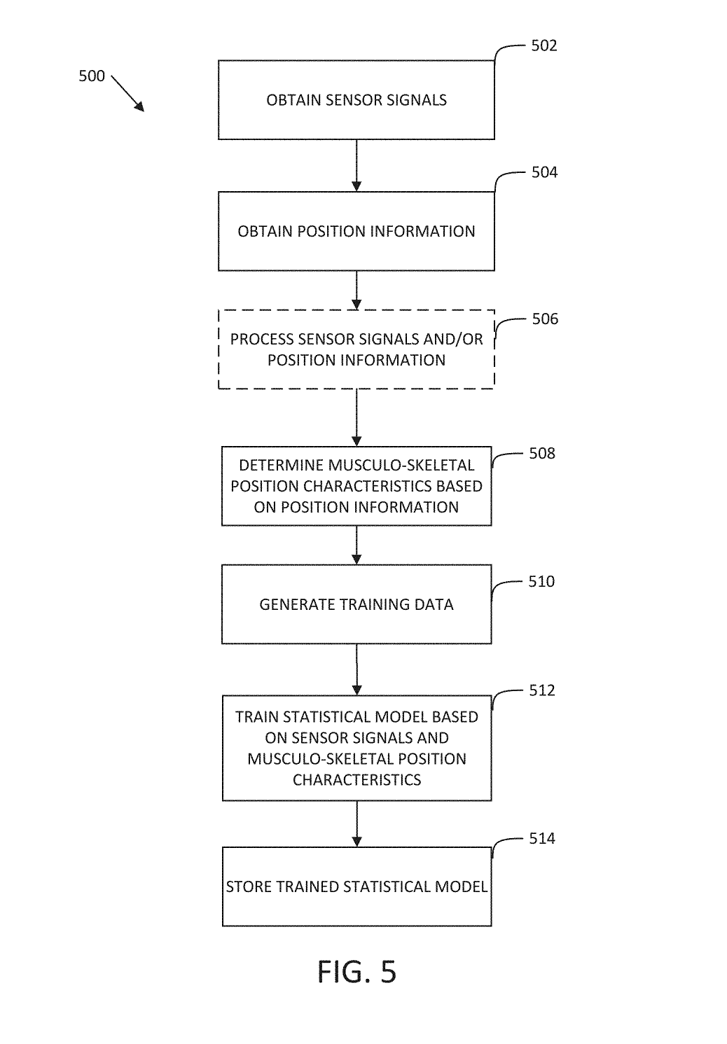

System 100 also includes one or more computer processors (not shown in FIG. 1) programmed to communicate with sensors 102. For example, signals recorded by one or more of the sensors may be provided to the processor(s), which may be programmed to execute one or more machine learning techniques that process signals output by the sensors 102 to train one or more statistical models 104, and the trained (or retrained) statistical model(s) 104 may be stored for later use in generating a musculoskeletal representation 106, as described in more detail below. Non-limiting examples of statistical models that may be used in accordance with some embodiments to predict handstate information based on recorded signals from sensors 102 are discussed in more detail below with regard to FIG. 5.

System 100 also optionally includes a display controller configured to display a visual representation 108 (e.g., of a hand). As discussed in more detail below, one or more computer processors may implement one or more trained statistical models configured to predict handstate information based, at least in part, on signals recorded by sensors 102. The predicted handstate information is used to update the musculoskeletal representation 106, which is then optionally used to render a visual representation 108 based on the updated musculoskeletal representation incorporating the current handstate information. Real-time reconstruction of the current handstate and subsequent rendering of the visual representation reflecting the current handstate information in the musculoskeletal model may provide visual feedback to the user about the effectiveness of the trained statistical model to accurately represent an intended handstate. Not all embodiments of system 100 include components configured to render a visual representation. For example, in some embodiments, handstate estimates output from the trained statistical model and a corresponding updated musculoskeletal representation are used to determine a state of a user's hand (e.g., in a virtual reality environment) even though a visual representation based on the updated musculoskeletal representation is not rendered (e.g., for interacting with virtual objects in a virtual environment in the absence of a virtually-rendered hand).

In some embodiments, a computer application configured to simulate a virtual reality environment may be instructed to display a visual representation of the user's hand. Positioning, movement, and/or forces applied by portions of the hand within the virtual reality environment may be displayed based on the output of the trained statistical model(s). The visual representation may be dynamically updated based on current reconstructed handstate information as continuous signals are recorded by the sensors 102 and processed by the trained statistical model(s) 104 to provide an updated computer-generated representation of the user's movement and/or exerted force that is updated in real-time.

As discussed above, some embodiments are directed to using a statistical model for predicting musculoskeletal information based on signals recorded from wearable autonomous sensors. The statistical model may be used to predict the musculoskeletal position information without having to place sensors on each segment of the rigid body that is to be represented in the computer-generated musculoskeletal representation. As discussed briefly above, the types of joints between segments in a multi-segment articulated rigid body model constrain movement of the rigid body. Additionally, different individuals tend to move in characteristic ways when performing a task that can be captured in statistical patterns of individual user behavior. At least some of these constraints on human body movement may be explicitly incorporated into statistical models used for prediction in accordance with some embodiments. Additionally or alternatively, the constraints may be learned by the statistical model through training based on ground truth data on the position and exerted forces of the hand and wrist in the context of recorded sensor data (e.g., EMG data). Constraints imposed in the construction of the statistical model are those set by anatomy and the physics of a user's body, while constraints derived from statistical patterns are those set by human behavior for one or more users from which sensor measurements are measured and used to train the statistical model. As described in more detail below, the constraints may comprise part of the statistical model itself being represented by information (e.g., connection weights between nodes) in the model.

As discussed above, some embodiments are directed to using a statistical model for predicting handstate information to enable the generation and/or real-time update of a computer-based musculoskeletal representation. The statistical model may be used to predict the handstate information based on IMU signals, neuromuscular signals (e.g., EMG, MMG, and SMG signals), external device signals (e.g., camera or laser-scanning signals), or a combination of IMU signals, neuromuscular signals, and external device signals detected as a user performs one or more movements.

Relative to tabletop research systems for neuromuscular recording, systems designed for independent use by a non-technical user, including wearable, wireless, and portable neuromuscular recording devices, are more susceptible to recording artifacts. Identifying, mitigating, and accounting for such artifacts is not trivial and doing so effectively enhances the accuracy of systems and methods for estimating the position, movement, and/or forces of a part of a user's body (e.g., hand).

The inventors have recognized that during continuous recording of neuromuscular signals from neuromuscular sensors, artifacts may occasionally appear in the recorded signal data for various reasons including, but not limited to, sensor malfunction and environmental factors such as 60 Hz noise. Providing neuromuscular signals including such artifacts as input to a trained statistical model as described above may result in inaccurate model output estimates (e.g., handstate estimates). The inventors have appreciated that detecting one or more artifacts in the continuously recorded neuromuscular signals in real-time and compensating for the detected artifacts prior to providing the neuromuscular signals as input to the trained statistical model may produce model estimates that more closely represent the user's movements.

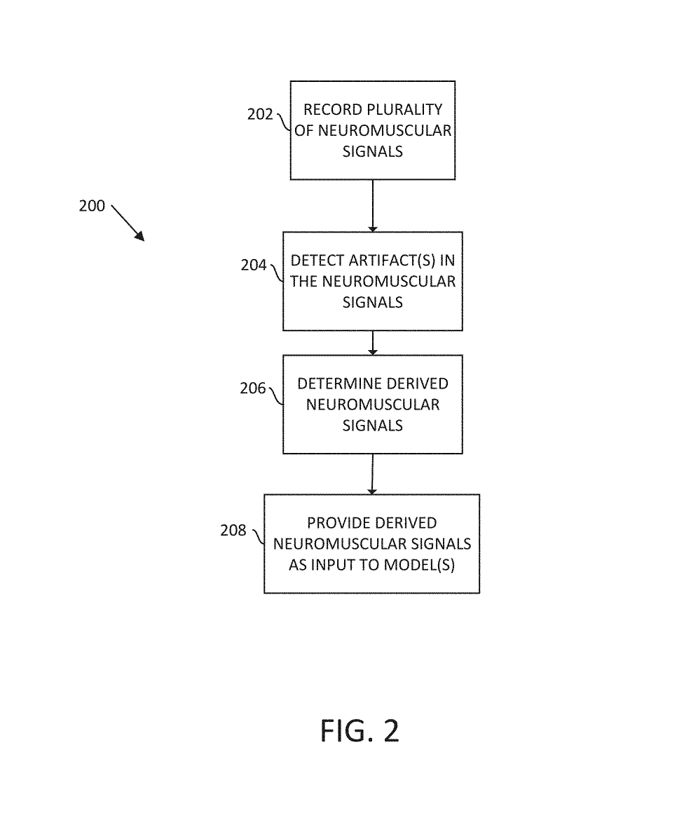

FIG. 2 illustrates a process 200 for detecting and mitigating artifacts in neuromuscular signal data in real-time in accordance with some embodiments. In act 202, a plurality of neuromuscular signals are recorded from a plurality of neuromuscular sensors. Process 200 proceeds to act 204, where the neuromuscular signals are analyzed to detect one or more artifacts in the neuromuscular signals in real-time as the neuromuscular signals are continuously recorded. Process 200 then proceeds to act 206, where derived neuromuscular signals are determined when one or more artifacts are detected in the neuromuscular signals. The derived neuromuscular signals are signals in which the detected artifacts have been mitigated by, for example, processing the signal data to at least partially remove the artifact(s) (e.g., via a filter or other suitable technique as described below), replacing at least some of the signal data with other signal data, or replacing the signal data with an average of signal data from neighboring sensors (as described below). Examples of determining derived neuromuscular signals are discussed in further detail below. Process 200 then proceeds to act 208, where the derived neuromuscular signals are provided as input to a trained statistical model in place of the recorded neuromuscular signals. In some instances, when no artifacts are detected in the neuromuscular signals recorded from particular sensors, the neuromuscular signals from those sensors may be provided as input to the trained statistical model without processing the signals to mitigate artifacts.

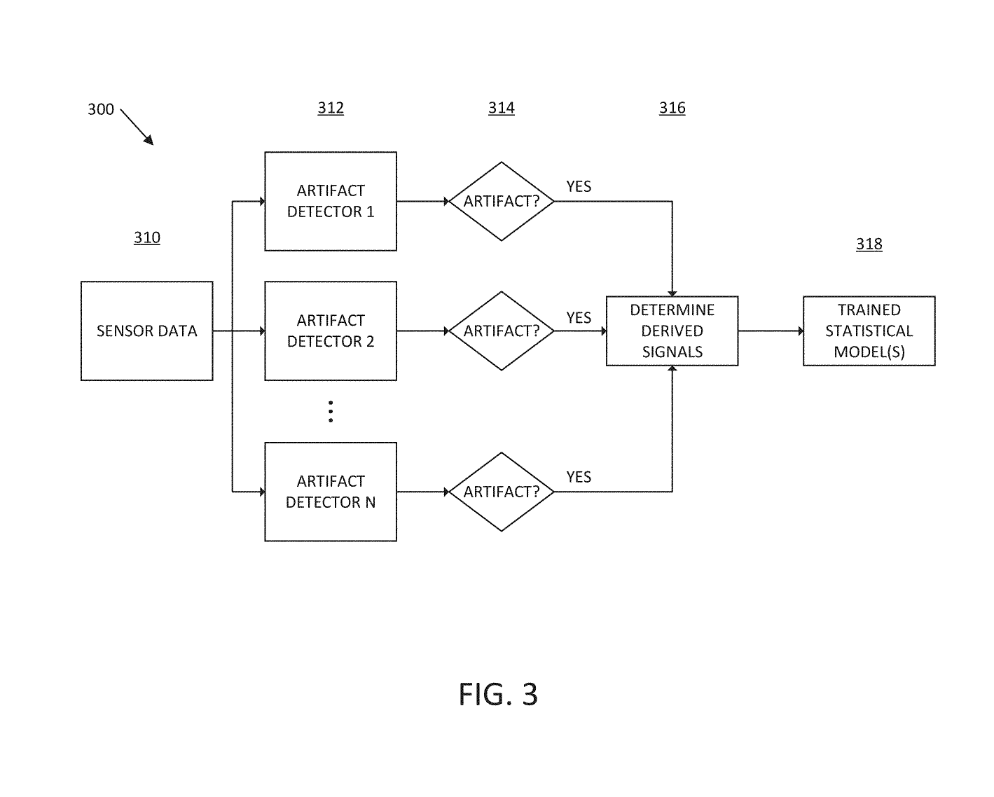

FIG. 3 illustrates a system architecture 300 for detecting and mitigating artifacts from recorded neuromuscular signals in accordance with some embodiments. Sensor data 310 recorded by a plurality of neuromuscular sensors is analyzed by a plurality of detector circuits 312, each of which is configured to detect a particular type of artifact by analyzing the sensor data. Detector circuits 312 may be implemented using hardware, software, or a combination of hardware and software (as described below). After analyzing the neuromuscular signals, a decision 314 is made as to whether the neuromuscular signals analyzed by the detector circuit 312 include the artifact for which the detector circuit is configured to detect. In some embodiments, decision 314 is made based, at least in part, on a quality metric associated with the neuromuscular signals analyzed by the detector circuit 312. For example, the quality metric may represent a probability (e.g., a confidence level) that the neuromuscular signals include a particular artifact, and it may be determined that the neuromuscular signals include the artifact when the probability is higher than a threshold value. Detector circuits 312 may be configured to process individual channels of neuromuscular sensor data or at least some of the detector circuits 312 may be configured to process neuromuscular sensor data recorded by multiple channels to detect artifacts. In an implementation where each of N detector circuits 312 is configured to process an individual channel of neuromuscular data to detect a particular artifact, the output of the artifact detection process may be a vector of N quality metrics, each of which corresponds to the analysis of the channel data by one of the detector circuits 312. Detector circuits 312 may be configured to detect any suitable signal artifact including, but not limited to, noise artifacts, skin-contact artifacts, skin lift-off artifacts, power line frequency (e.g., 50 Hz, 60 Hz) artifacts, clipped signal artifacts, inactive sensor artifacts, microfriction artifacts, data degeneration artifacts, and artifacts caused by movement of one or more neuromuscular sensors (e.g., the rotation of an armband containing a plurality of neuromuscular sensors that causes the mapping between the location of one or more neuromuscular sensors and the recorded signals of the neuromuscular sensors generated by underlying motor units to change).

When decision 314 indicates that the neuromuscular sensor data includes an artifact detected by a corresponding detector circuit 312, one or more derived neuromuscular signals 316 are determined in which the artifact has been mitigated by, for example, at least partially removing the artifact or replacing the signals with other signals. The derived neuromuscular signals may be determined in any suitable way based on the decisions 314 output from the detector circuits 312 and one or more rules associated with those decisions. In some embodiments, information output from the detector circuits is used to determine whether to process the neuromuscular signals to mitigate the detected artifact(s) or whether to replace the neuromuscular sensor data with other sensor data or data derived from other sensor data.

The decision on whether to process the sensor data or replace the data may be made in any suitable way. In some embodiments, the decision of whether to process or replace the sensor data may be made on a detector circuit by detector circuit basis. For example, for some detector circuits, the neuromuscular signals may always be processed (rather than replaced) to mitigate a detected artifact based on the type of artifact that the detector circuit is configured to detect. For example, if the artifact detected is external 60 Hz noise, the neuromuscular signals may always be processed by filtering rather than being replaced. In other instances, the neuromuscular signals may always be replaced rather than being processed. For example, if the detector circuit is configured to detect artifacts corresponding to a disconnected or malfunctioning sensor, the neuromuscular signals may always be replaced (rather than being processed) with an average (or other metric) of neuromuscular signals from one or more neighboring sensors. In yet other instances, a determination of whether the neuromuscular signals analyzed by a particular detector circuit should be processed or replaced is made based, at least in part, on a quality factor determined as a result of the analysis by the detector circuit. For example, if the quality factor is less than a threshold value or within a first range, the neuromuscular sensor data may be replaced, whereas when the quality factor is greater than a threshold or within a second range, the neuromuscular sensor data may be processed.

In some embodiments, the decision on whether to process neuromuscular signals with detected artifact(s) or replace the neuromuscular signals may be made based on the output of multiple detector circuits. For example, if the detector circuits indicate that multiple artifacts in a particular neuromuscular sensor channel or group of neuromuscular sensors have been detected, it may be determined to replace the neuromuscular signals due to the poor quality of the recorded signals.

When it is determined to process the neuromuscular signals based on the decision 314 for a particular detector circuit 312, the processing may be performed in any suitable way to mitigate the detected artifact. For example, the neuromuscular signals may be filtered or otherwise processed to mitigate the detected artifact. The type of artifact and/or characteristics of the artifact that is detected may inform how the neuromuscular signals are processed. In some implementations the neuromuscular signals may be analyzed to determine one or more characteristics of the artifact by, for example, calculating a power spectrum to determine the frequency characteristics of the artifact, or fitting a generative model of certain artifact types to the neuromuscular signals. After determining the artifact characteristic(s) the neuromuscular signals may be processed to mitigate the artifact. For some types of artifacts (e.g., skin lift-off artifacts), the processing may involve filtering techniques (e.g., a high pass filter above a critical frequency). For other types of artifacts, the processing may involve subtracting at least some estimated artifact behavior from the recorded neuromuscular signals using, for example, a generative model.

When it is determined to replace the neuromuscular signals based on the decision 314 for a particular detector circuit 312 or collection of detector circuits 312, the replacing may be performed in any suitable way to mitigate the detected artifact. For example, if the detected artifact occurs over a relatively short time period, the neuromuscular signal data for a particular sensor may be replaced with signal data from the same sensor recorded at an earlier point in time when the artifact was not present in the signal. Replacing the corrupted signal data (e.g., signal data with detected artifacts) with signal data from the same sensor may be preferred in some instances because the signal data used for replacement has been recorded for neuromuscular activity from the same muscle or muscles as the corrupted signal data. Alternatively, if the detected artifact occurs over a relatively long period of time or if the signal data from the sensor is unusable (e.g., if the sensor has been disconnected or has contact issues), the signal data may be replaced with signal data recorded by other sensors. For example, the signal data may be replaced based on signal data recorded by one or more sensors arranged adjacent to the sensor having the corrupted data. In some embodiments, the signal data for the corrupted sensor may be replaced with an average of signal data from two or more neighboring sensors to the corrupted sensor. For example, the two or more neighboring sensors may be arranged next to or near the corrupted sensor on a wearable device that includes the plurality of neuromuscular sensors. In some embodiments, the average of signal data may be a weighted average of signal data, where the weights are determined in any suitable way. In some embodiments, the weight for data recorded by a neuromuscular sensor with an artifact may be set to zero such that data from that sensor is not considered. In some embodiments, signal data from a neuromuscular sensor with an artifact may be imputed based on neuromuscular signal data derived from historical data of neuromuscular sensors in an array of neuromuscular sensors that are not experiencing an artifact. In certain embodiments, imputed signal data may be user-specific or based on data from a population of users. Signal data from a neuromuscular sensor experiencing an artifact may be inferred and the inference may comprise raw signal data or processed signal data (e.g., amplitude, cospectrum matrix, or another metric). In some embodiments, the inference about signal data from a neuromuscular sensor experiencing an artifact may be generated based on one or more of: general constraints about the neuromuscular system and human anatomy; personal constraints related to the user's physiology and/or anatomy; and session-specific constraints related to the particular positioning and impedance of a plurality of neuromuscular sensors.

When only a single detector circuit 312 of a plurality of detector circuits detects an artifact in the analyzed neuromuscular signals, the neuromuscular signals may be processed or replaced based on one or more rules specifying how to process/replace data when the artifact is detected by the single detector circuit 312. When multiple detector circuits detect artifacts in the analyzed neuromuscular signals, the neuromuscular signals may be processed or replaced based on one or more rules specifying how to process or replace data when multiple artifacts are detected. For example, the one or more rules may specify a processing hierarchy (or order) based on the detected artifacts such that processing to mitigate certain artifacts is performed prior to processing to mitigate other artifacts. Additionally, the one or more rules may specify that if any of the detector circuits 312 detects an artifact with a quality value less than a particular threshold, the signal data is replaced (rather than processed) regardless of artifacts detected by the other detector circuits 312. Any other rules may alternatively be used, and embodiments are not limited in this respect.

After the derived signals 316 in which the signal artifacts have been mitigated are determined, the derived signals are provided as input to a trained statistical model 318, which in turn is configured to output estimates (e.g., handstate estimates) based on the input signals. It should be appreciated that some neuromuscular signals may be processed/replaced when an artifact is detected, whereas other contemporaneously recorded neuromuscular signals (e.g., from other sensors) may not be processed/replaced when no artifacts are detected, and the combination of unprocessed (for mitigating artifacts) and derived signals may be provided as input to the trained statistical model.

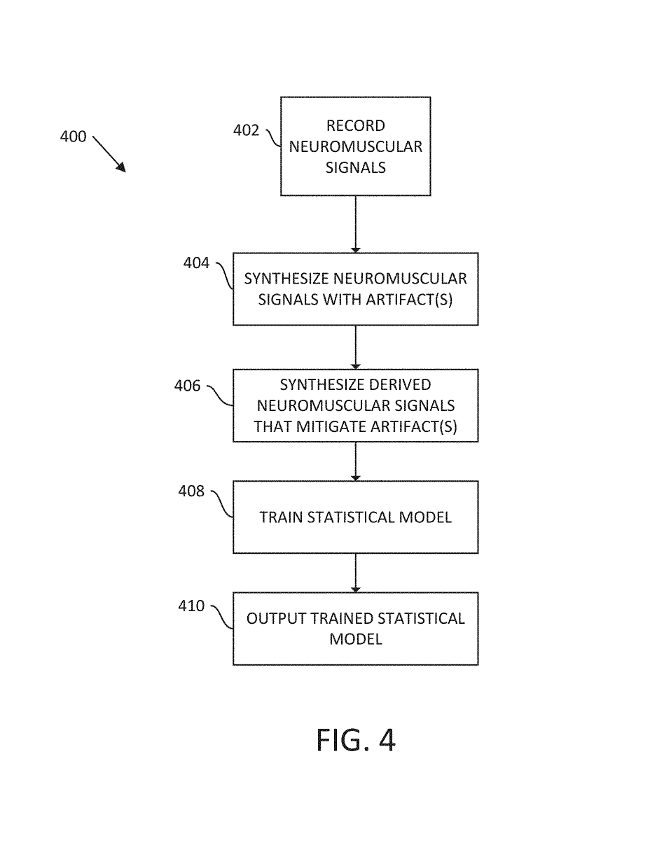

The inventors have recognized that trained statistical models used in accordance with some embodiments may be trained using training data that makes the model more robust to artifacts in the recorded signals. FIG. 4 illustrates a process 400 for training a statistical model using training data that includes neuromuscular signals associated with artifacts. The artifacts may be recorded as part of the neuromuscular signals or the artifacts may be simulated and added to the recorded neuromuscular signals. Such a trained statistical model when used during runtime may be more robust to artifacts in the recorded neuromuscular signals. Process 400 begins in act 402 where neuromuscular signals are continuously recorded. Process 400 then proceeds to act 404 where neuromuscular signals with one or more artifacts are synthesized by modifying the recorded neuromuscular signals to include characteristics of the artifact. The synthesized neuromuscular signals may be created in any suitable way. For example, noise may be added to the neuromuscular signals to simulate the presence of noise in the recorded signals when used in a particular environment (e.g., an environment in which 60 Hz noise is prevalent). In some embodiments, synthesized neuromuscular signals based on some or all of the types of artifacts detected by the detection circuits described in connection with the architecture of FIG. 3 may be used in act 404. Alternatively, the recorded neuromuscular signals used as training data to train the model may already have artifacts included in the recorded signals, making it unnecessary to simulate the artifacts and add the simulated artifacts into “clean” neuromuscular signals. For example, an armband with a plurality of neuromuscular sensors may be worn loosely in order to generate frequent contact artifacts caused by a neuromuscular sensor transiently losing low-impedance contact with the skin.

Process 400 then proceeds to act 406, where derived neuromuscular signals are synthesized in which the artifacts introduced to the neuromuscular signals in act 404 have been mitigated. Realizing that the mitigation techniques described herein for mitigating signal artifacts may not entirely remove the signal artifacts, inclusion in the training data of synthesized derived neuromuscular signal data that mimics the operation of the mitigation techniques used during runtime results in a trained statistical model that may provide more accurate model estimates.

Process 400 then proceeds to act 408, where the statistical model is trained using training data that includes the synthesized derived neuromuscular signals. Following training, process 400 proceeds to act 410, where the trained statistical model is output for use during runtime as described above in connection with FIGS. 1-3.

In some embodiments, a denoising autoencoder as a component of a statistical model is used to identify and mitigate an artifact. A denoising autoencoder can be implemented by building a statistical model (e.g., a neural network) where input data comprises clean neuromuscular sensor data containing no (or few) artifacts combined with artifacts (e.g., noise), then training the model with the clean neuromuscular sensor data. In this manner, a system or method with a statistical model comprising a denoising autoencoder may provide robustness to neuromuscular artifacts. The artifacts added to the clean neuromuscular sensor data may have a statistical structure consistent with any suitable signal artifact including, but not limited to, noise artifacts, skin-contact artifacts, skin lift-off artifacts, power line frequency (e.g., 50 Hz, 60 Hz) artifacts, clipped signal artifacts, inactive sensor artifacts, microfriction artifacts, data degeneration artifacts, and artifacts caused by movement of one or more neuromuscular sensors (e.g., the rotation of an armband containing a plurality of neuromuscular sensors that causes the mapping between the location of one or more neuromuscular sensors and the recorded signals of the neuromuscular sensors generated by underlying motor units to change).

In some embodiments, simple quality metrics may be derived from the first few principal components of the log power spectra of the neuromuscular sensor data, which tend to be stereotyped across electrodes and between users and recording sessions. For example, linear and quadratic discriminant analysis may account for common causes of aberrant power spectra (e.g., to be able to identify artifacts including but not limited to: motion artifacts (low frequency), contact artifacts (broadband noise), power-line noise (60 Hz), artifacts caused by ground truth data systems that determine the position of a part of the user's body (e.g. joints of the hand), and IMU artifacts). In a variation of this embodiment, cospectral features of multi-channel neuromuscular data may be used to identify artifacts manifesting as correlational information between neuromuscular sensors.

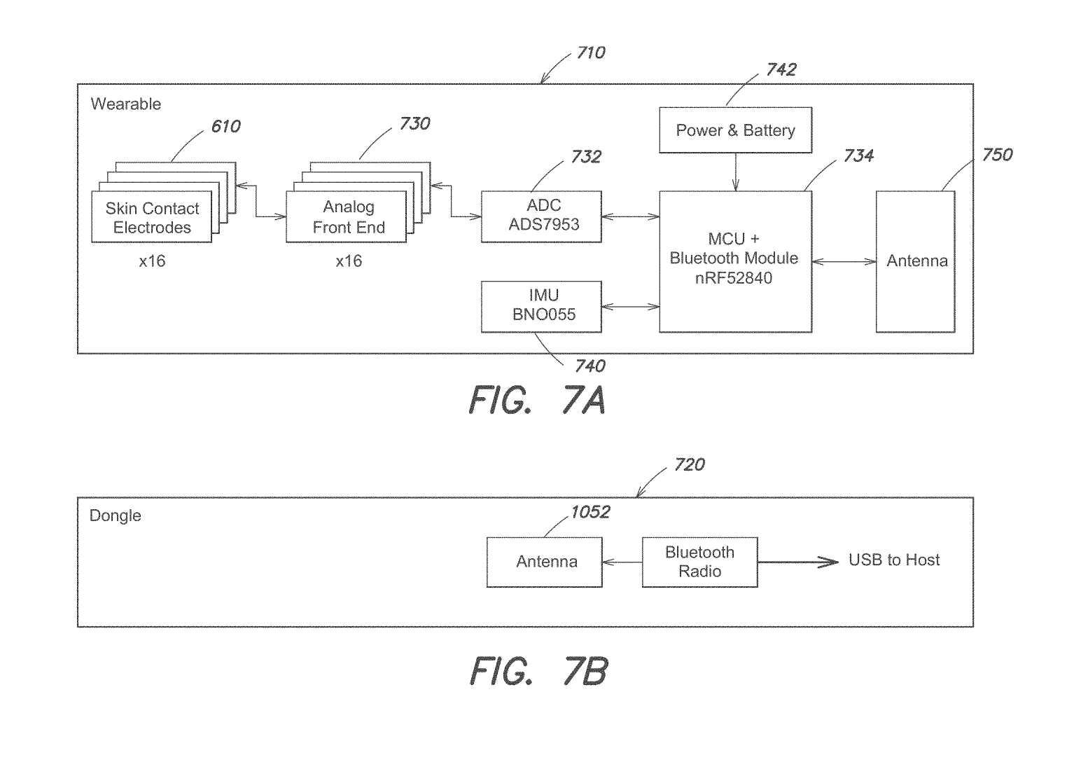

FIG. 5 describes a process 500 for generating (sometimes termed “training” herein) a statistical model using signals recorded from sensors 102. Process 500 may be executed by any suitable computing device(s), as aspects of the technology described herein are not limited in this respect. For example, process 500 may be executed by one or more computer processors described with reference to FIGS. 7A and 7B. As another example, one or more acts of process 500 may be executed using one or more servers (e.g., servers included as a part of a cloud computing environment). For example, at least a portion of act 510 relating to training of a statistical model (e.g., a neural network) may be performed using a cloud computing environment.

Process 500 begins at act 502, where a plurality of sensor signals are obtained for one or multiple users performing one or more movements (e.g., typing on a keyboard). In some embodiments, the plurality of sensor signals may be recorded as part of process 500. In other embodiments, the plurality of sensor signals may have been recorded prior to the performance of process 500 and are accessed (rather than recorded) at act 502.

In some embodiments, the plurality of sensor signals may include sensor signals recorded for a single user performing a single movement or multiple movements. The user may be instructed to perform a sequence of movements for a particular task (e.g., opening a door) and sensor signals corresponding to the user's movements may be recorded as the user performs the task he/she was instructed to perform. The sensor signals may be recorded by any suitable number of sensors located in any suitable location(s) to detect the user's movements that are relevant to the task performed. For example, after a user is instructed to perform a task with the fingers of his/her right hand, the sensor signals may be recorded by multiple neuromuscular sensors circumferentially (or otherwise) arranged around the user's lower right arm to detect muscle activity in the lower right arm that give rise to the right hand movements and one or more IMU sensors arranged to predict the joint angle of the user's arm relative to the user's torso. As another example, after a user is instructed to perform a task with his/her leg (e.g., to kick an object), sensor signals may be recorded by multiple neuromuscular sensors circumferentially (or otherwise) arranged around the user's leg to detect muscle activity in the leg that give rise to the movements of the foot and one or more IMU sensors arranged to predict the joint angle of the user's leg relative to the user's torso.

In some embodiments, the sensor signals obtained in act 502 correspond to signals from one type of sensor (e.g., one or more IMU sensors or one or more neuromuscular sensors) and a statistical model may be trained based on the sensor signals recorded using the particular type of sensor, resulting in a sensor-type specific trained statistical model. For example, the obtained sensor signals may comprise a plurality of EMG sensor signals arranged around the lower arm or wrist of a user and the statistical model may be trained to predict musculoskeletal position information for movements of the wrist and/or hand during performance of a task such as grasping and twisting an object such as a doorknob.

In embodiments that provide predictions based on multiple types of sensors (e.g., IMU sensors, EMG sensors, MMG sensors, SMG sensors), a separate statistical model may be trained for each of the types of sensors and the outputs of the sensor-type specific models may be combined to generate a musculoskeletal representation of the user's body. In other embodiments, the sensor signals obtained in act 502 from two or more different types of sensors may be provided to a single statistical model that is trained based on the signals recorded from the different types of sensors. In one illustrative implementation, an IMU sensor and a plurality of EMG sensors are arranged on a wearable device configured to be worn around the forearm of a user, and signals recorded by the IMU and EMG sensors are collectively provided as inputs to a statistical model, as discussed in more detail below.

In some embodiments, the sensor signals obtained in act 502 are recorded at multiple time points as a user performs one or multiple movements. As a result, the recorded signal for each sensor may include data obtained at each of multiple time points. Assuming that n sensors are arranged to simultaneously measure the user's movement information during performance of a task, the recorded sensor signals for the user may comprise a time series of K n-dimensional vectors {xₖ|1≤k≤K} at time points t₁, t₂, . . . , t_{K} during performance of the movements.

In some embodiments, a user may be instructed to perform a task multiple times and the sensor signals and position information may be recorded for each of multiple repetitions of the task by the user. In some embodiments, the plurality of sensor signals may include signals recorded for multiple users, each of the multiple users performing the same task one or more times. Each of the multiple users may be instructed to perform the task and sensor signals and position information corresponding to that user's movements may be recorded as the user performs (once or repeatedly) the task he/she was instructed to perform. When sensor signals are collected by multiple users which are combined to generate a statistical model, an assumption is that different users employ similar musculoskeletal positions to perform the same movements. Collecting sensor signals and position information from a single user performing the same task repeatedly and/or from multiple users performing the same task one or multiple times facilitates the collection of sufficient training data to generate a statistical model that can accurately predict musculoskeletal position information associated with performance of the task.

In some embodiments, a user-independent statistical model may be generated based on training data corresponding to the recorded signals from multiple users, and as the system is used by a user, the statistical model is trained based on recorded sensor data such that the statistical model learns the user-dependent characteristics to refine the prediction capabilities of the system for the particular user.

In some embodiments, the plurality of sensor signals may include signals recorded for a user (or each of multiple users) performing each of multiple tasks one or multiple times. For example, a user may be instructed to perform each of multiple tasks (e.g., grasping an object, pushing an object, and pulling open a door) and signals corresponding to the user's movements may be recorded as the user performs each of the multiple tasks he/she was instructed to perform. Collecting such data may facilitate developing a statistical model for predicting musculoskeletal position information associated with multiple different actions that may be taken by the user. For example, training data that incorporates musculoskeletal position information for multiple actions may facilitate generating a statistical model for predicting which of multiple possible movements a user may be performing.

As discussed above, the sensor data obtained at act 502 may be obtained by recording sensor signals as each of one or multiple users performs each of one or more tasks one or more multiple times. As the user(s) perform the task(s), position information describing the spatial position of different body segments during performance of the task(s) may be obtained in act 504. In some embodiments, the position information is obtained using one or more external devices or systems that track the position of different points on the body during performance of a task. For example, a motion capture system, a laser scanner, a device to measure mutual magnetic induction, or some other system configured to capture position information may be used. As one non-limiting example, a plurality of position sensors may be placed on segments of the fingers of the right hand and a motion capture system may be used to determine the spatial location of each of the position sensors as the user performs a task such as grasping an object. The sensor data obtained at act 502 may be recorded simultaneously with recording of the position information obtained in act 504. In this example, position information indicating the position of each finger segment over time as the grasping motion is performed is obtained.

Next, process 500 proceeds to act 506, where the sensor signals obtained in act 502 and/or the position information obtained in act 504 are optionally processed. For example, the sensor signals or the position information signals may be processed using amplification, filtering, rectification, or other types of signal processing.

Next, process 500 proceeds to act 508, where musculoskeletal position characteristics are determined based on the position information (as collected in act 504 or as processed in act 506). In some embodiments, rather than using recorded spatial (e.g., x, y, z) coordinates corresponding to the position sensors as training data to train the statistical model, a set of derived musculoskeletal position characteristic values are determined based on the recorded position information, and the derived values are used as training data for training the statistical model. For example, using information about the constraints between connected pairs of rigid segments in the articulated rigid body model, the position information may be used to determine joint angles that define angles between each connected pair of rigid segments at each of multiple time points during performance of a task. Accordingly, the position information obtained in act 504 may be represented by a vector of n joint angles at each of a plurality of time points, where n is the number of joints or connections between segments in the articulated rigid body model.

Next, process 500 proceeds to act 510, where the time series information obtained at acts 502 and 508 is combined to create training data used for training a statistical model at act 510. The obtained data may be combined in any suitable way. In some embodiments, each of the sensor signals obtained at act 502 may be associated with a task or movement within a task corresponding to the musculoskeletal position characteristics (e.g., joint angles) determined based on the positional information recorded in act 504 as the user performed the task or movement. In this way, the sensor signals may be associated with musculoskeletal position characteristics (e.g., joint angles) and the statistical model may be trained to predict that the musculoskeletal representation will be characterized by particular musculoskeletal position characteristics between different body segments when particular sensor signals are recorded during performance of a particular task.

In embodiments comprising sensors of different types (e.g., IMU sensors and neuromuscular sensors) configured to simultaneously record different types of movement information during performance of a task, the sensor data for the different types of sensors may be recorded using the same or different sampling rates. When the sensor data is recorded at different sampling rates, at least some of the sensor data may be resampled (e.g., up-sampled or down-sampled) such that all sensor data provided as input to the statistical model corresponds to time series data at the same time resolution. Resampling at least some of the sensor data may be performed in any suitable way including, but not limited to using interpolation for upsampling and using decimation for downsampling.

In addition to or as an alternative to resampling at least some of the sensor data when recorded at different sampling rates, some embodiments employ a statistical model configured to accept multiple inputs asynchronously. For example, the statistical model may be configured to model the distribution of the “missing” values in the input data having a lower sampling rate. Alternatively, the timing of training of the statistical model occur asynchronously as input from multiple sensor data measurements becomes available as training data.

Next, process 500 proceeds to act 512, where a statistical model for predicting musculoskeletal position information is trained using the training data generated at act 510. The statistical model being trained may take as input a sequence of data sets each of the data sets in the sequence comprising an n-dimensional vector of sensor data. The statistical model may provide output that indicates, for each of one or more tasks or movements that may be performed by a user, the likelihood that the musculoskeletal representation of the user's body will be characterized by a set of musculoskeletal position characteristics (e.g., a set of joint angles between segments in an articulated multi-segment body model). For example, the statistical model may take as input a sequence of vectors {xₖ|1≤k≤K} generated using measurements obtained at time points t₁, t₂, . . . , t_{K}, where the ith component of vector xⱼ is a value measured by the ith sensor at time t₃ and/or derived from the value measured by the ith sensor at time t₃. In another non-limiting example, a derived value provided as input to the statistical model may comprise features extracted from the data from all or a subset of the sensors at and/or prior to time tⱼ (e.g., a covariance matrix, a power spectrum, a combination thereof, or any other suitable derived representation). Based on such input, the statistical model may provide output indicating, a probability that a musculoskeletal representation of the user's body will be characterized by a set of musculoskeletal position characteristics. As one non-limiting example, the statistical model may be trained to predict a set of joint angles for segments in the fingers in the hand over time as a user grasps an object. In this example, the trained statistical model may output, a set of predicted joint angles for joints in the hand corresponding to the sensor input.

In some embodiments, the statistical model may be a neural network and, for example, may be a recurrent neural network. In some embodiments, the recurrent neural network may be a long short-term memory (LSTM) neural network. It should be appreciated, however, that the recurrent neural network is not limited to being an LSTM neural network and may have any other suitable architecture. For example, in some embodiments, the recurrent neural network may be a fully recurrent neural network, a recursive neural network, a variational autoencoder, a Hopfield neural network, an associative memory neural network, an Elman neural network, a Jordan neural network, an echo state neural network, a second order recurrent neural network, and/or any other suitable type of recurrent neural network. In other embodiments, neural networks that are not recurrent neural networks may be used. For example, deep neural networks, convolutional neural networks, and/or feedforward neural networks, may be used.

In some of the embodiments in which the statistical model is a neural network, the output layer of the neural network may provide a set of output values corresponding to a respective set of possible musculoskeletal position characteristics (e.g., joint angles). In this way, the neural network may operate as a non-linear regression model configured to predict musculoskeletal position characteristics from raw or pre-processed sensor measurements. It should be appreciated that, in some embodiments, any other suitable non-linear regression model may be used instead of a neural network, as aspects of the technology described herein are not limited in this respect.

In some embodiments, the neural network can be implemented based on a variety of topologies and/or architectures including deep neural networks with fully connected (dense) layers, Long Short-Term Memory (LSTM) layers, convolutional layers, Temporal Convolutional Layers (TCL), or other suitable type of deep neural network topology and/or architecture. The neural network can have different types of output layers including output layers with logistic sigmoid activation functions, hyperbolic tangent activation functions, linear units, rectified linear units, or other suitable type of nonlinear unit. Likewise, the neural network can be configured to represent the probability distribution over n different classes via, for example, a softmax function or include an output layer that provides a parameterized distribution e.g., mean and variance of a Gaussian distribution.

It should be appreciated that aspects of the technology described herein are not limited to using neural networks, as other types of statistical models may be employed in some embodiments. For example, in some embodiments, the statistical model may comprise a hidden Markov model, a Markov switching model with the switching allowing for toggling among different dynamic systems, dynamic Bayesian networks, and/or any other suitable graphical model having a temporal component. Any such statistical model may be trained at act 512 using the sensor data obtained at act 502.

As another example, in some embodiments, the statistical model may take as input, features derived from the sensor data obtained at act 502. In such embodiments, the statistical model may be trained at act 512 using features extracted from the sensor data obtained at act 502. The statistical model may be a support vector machine, a Gaussian mixture model, a regression based classifier, a decision tree classifier, a Bayesian classifier, and/or any other suitable classifier, as aspects of the technology described herein are not limited in this respect. Input features to be provided as training data to the statistical model may be derived from the sensor data obtained at act 502 in any suitable way. For example, the sensor data may be analyzed as time series data using wavelet analysis techniques (e.g., continuous wavelet transform, discrete-time wavelet transform, etc.), Fourier-analytic techniques (e.g., short-time Fourier transform, Fourier transform, etc.), and/or any other suitable type of time-frequency analysis technique. As one non-limiting example, the sensor data may be transformed using a wavelet transform and the resulting wavelet coefficients may be provided as inputs to the statistical model.

In some embodiments, at act 512, values for parameters of the statistical model may be estimated from the training data generated at act 510. For example, when the statistical model is a neural network, parameters of the neural network (e.g., weights) may be estimated from the training data. In some embodiments, parameters of the statistical model may be estimated using gradient descent, stochastic gradient descent, and/or any other suitable iterative optimization technique. In embodiments where the statistical model is a recurrent neural network (e.g., an LSTM), the statistical model may be trained using stochastic gradient descent and backpropagation through time. The training may employ a cross-entropy loss function and/or any other suitable loss function, as aspects of the technology described herein are not limited in this respect.

Next, process 500 proceeds to act 514, where the trained statistical model is stored (e.g., in datastore—not shown). The trained statistical model may be stored using any suitable format, as aspects of the technology described herein are not limited in this respect. In this way, the statistical model generated during execution of process 500 may be used at a later time, for example, to predict musculoskeletal position information (e.g., joint angles) for a given set of input sensor data, as described below.

In some embodiments, sensor signals are recorded from a plurality of sensors (e.g., arranged on or near the surface of a user's body) that record activity associated with movements of the body during performance of a task. The recorded signals may be optionally processed and provided as input to a statistical model trained using one or more techniques described above in connection with FIG. 5. In some embodiments that continuously record autonomous signals, the continuously recorded signals (raw or processed) may be continuously or periodically provided as input to the trained statistical model for prediction of musculoskeletal position information (e.g., joint angles) for the given set of input sensor data. As discussed above, in some embodiments, the trained statistical model is a user-independent model trained based on autonomous sensor and position information measurements from a plurality of users. In other embodiments, the trained model is a user-dependent model trained on data recorded from the individual user from which the data associated with the sensor signals is also acquired.

After the trained statistical model receives the sensor data as a set of input parameters, the predicted musculoskeletal position information is output from the trained statistical model. As discussed above, in some embodiments, the predicted musculoskeletal position information may comprise a set of musculoskeletal position information values (e.g., a set of joint angles) for a multi-segment articulated rigid body model representing at least a portion of the user's body. In other embodiments, the musculoskeletal position information may comprise a set of probabilities that the user is performing one or more movements from a set of possible movements.

In some embodiments, after musculoskeletal position information is predicted, a computer-based musculoskeletal representation of the user's body is generated based, at least in part, on the musculoskeletal position information output from the trained statistical model. The computer-based musculoskeletal representation may be generated in any suitable way. For example, a computer-based musculoskeletal model of the human body may include multiple rigid body segments, each of which corresponds to one or more skeletal structures in the body. For example, the upper arm may be represented by a first rigid body segment, the lower arm may be represented by a second rigid body segment the palm of the hand may be represented by a third rigid body segment, and each of the fingers on the hand may be represented by at least one rigid body segment (e.g., at least fourth-eighth rigid body segments). A set of joint angles between connected rigid body segments in the musculoskeletal model may define the orientation of each of the connected rigid body segments relative to each other and a reference frame, such as the torso of the body. As new sensor data is measured and processed by the statistical model to provide new predictions of the musculoskeletal position information (e.g., an updated set of joint angles), the computer-based musculoskeletal representation of the user's body may be updated based on the updated set of joint angles determined based on the output of the statistical model. In this way the computer-based musculoskeletal representation is dynamically updated in real-time as sensor data is continuously recorded.

The computer-based musculoskeletal representation may be represented and stored in any suitable way, as embodiments of the technology described herein are not limited with regard to the particular manner in which the representation is stored. Additionally, although referred to herein as a “musculoskeletal” representation, to reflect that muscle activity may be associated with the representation in some embodiments, as discussed in more detail below, it should be appreciated that some musculoskeletal representations used in accordance with some embodiments may correspond to skeletal structures, muscular structures or a combination of skeletal structures and muscular structures in the body.

In some embodiments, direct measurement of neuromuscular activity and/or muscle activity underlying the user's movements may be combined with the generated musculoskeletal representation. Measurements from a plurality of sensors placed at locations on a user's body may be used to create a unified representation of muscle recruitment by superimposing the measurements onto a dynamically-posed skeleton. In some embodiments, muscle activity sensed by neuromuscular sensors and/or information derived from the muscle activity (e.g., force information) may be combined with the computer-generated musculoskeletal representation in real time.

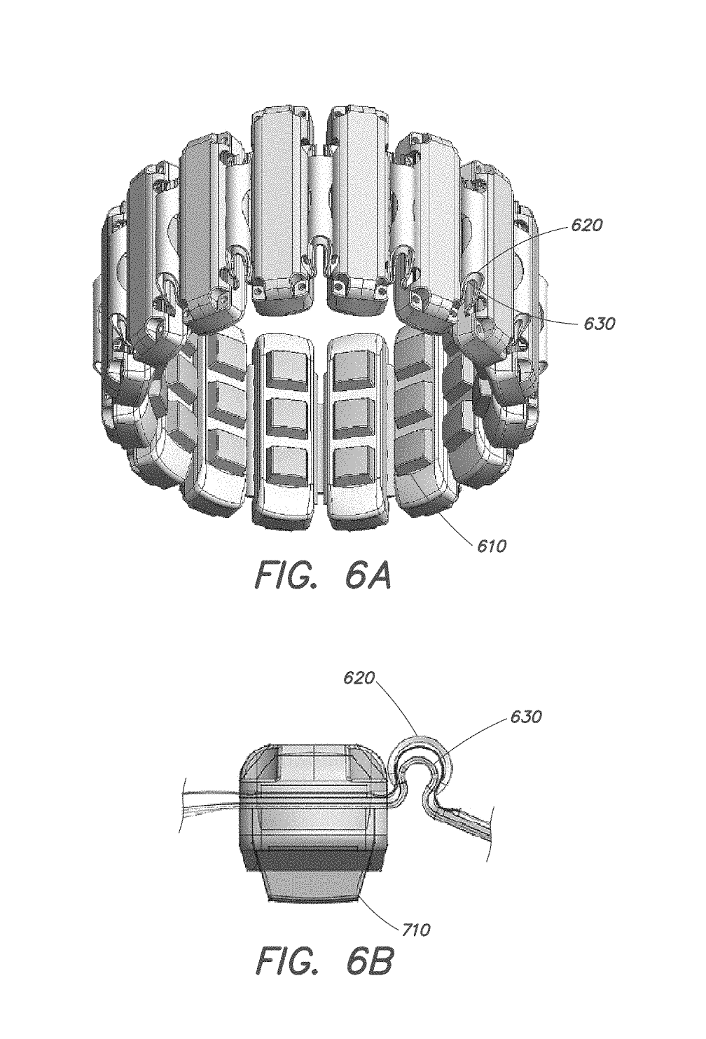

FIG. 6A illustrates a wearable system with sixteen neuromuscular sensors 610 (e.g., EMG sensors) arranged circumferentially around an elastic band 620 configured to be worn around a user's lower arm or wrist. As shown, EMG sensors 610 are arranged circumferentially around elastic band 620. It should be appreciated that any suitable number of neuromuscular sensors may be used. The number and arrangement of neuromuscular sensors may depend on the particular application for which the wearable device is used. For example, a wearable armband or wristband can be used to generate control information for controlling an augmented reality system, a robot, controlling a vehicle, scrolling through text, controlling a virtual avatar, or any other suitable control task.

In some embodiments, sensors 610 includes a set of neuromuscular sensors (e.g., EMG sensors). In other embodiments, sensors 610 can include a set of neuromuscular sensors and at least one “auxiliary” sensor configured to continuously record auxiliary signals. Examples of auxiliary sensors include, but are not limited to, other sensors such as IMU sensors, microphones, imaging sensors (e.g., a camera), radiation based sensors for use with a radiation-generation device (e.g., a laser-scanning device), or other types of sensors such as a heart-rate monitor. As shown the sensors 610 may be coupled together using flexible electronics 630 incorporated into the wearable device. FIG. 6B illustrates a cross-sectional view through one of the sensors 610 of the wearable device shown in FIG. 6A.

In some embodiments, the output of one or more of the sensing components can be optionally processed using hardware signal processing circuitry (e.g., to perform amplification, filtering, and/or rectification). In other embodiments, at least some signal processing of the output of the sensing components can be performed in software. Thus, signal processing of signals sampled by the sensors can be performed in hardware, software, or by any suitable combination of hardware and software, as aspects of the technology described herein are not limited in this respect. A non-limiting example of a signal processing chain used to process recorded data from sensors 610 is discussed in more detail below in connection with FIGS. 7A and 7B

FIGS. 7A and 7B illustrate a schematic diagram with internal components of a wearable system with sixteen EMG sensors, in accordance with some embodiments of the technology described herein. As shown, the wearable system includes a wearable portion 710 (FIG. 7A) and a dongle portion 720 (FIG. 7B) in communication with the wearable portion 710 (e.g., via Bluetooth or another suitable short range wireless communication technology). As shown in FIG. 7A, the wearable portion 710 includes the sensors 610, examples of which are described in connection with FIGS. 6A and 6B. The output of the sensors 610 is provided to analog front end 730 configured to perform analog processing (e.g., noise reduction, filtering, etc.) on the recorded signals. The processed analog signals are then provided to analog-to-digital converter 732, which converts the analog signals to digital signals that can be processed by one or more computer processors. An example of a computer processor that may be used in accordance with some embodiments is microcontroller (MCU) 734 illustrated in FIG. 7A. As shown, MCU 734 may also include inputs from other sensors (e.g., IMU sensor 740), and power and battery module 742. The output of the processing performed by MCU may be provided to antenna 750 for transmission to dongle portion 720 shown in FIG. 7B.

Dongle portion 720 includes antenna 752 configured to communicate with antenna 750 included as part of wearable portion 710. Communication between antenna 750 and 752 may occur using any suitable wireless technology and protocol, non-limiting examples of which include radiofrequency signaling and Bluetooth. As shown, the signals received by antenna 752 of dongle portion 720 may be provided to a host computer for further processing, display, and/or for effecting control of a particular physical or virtual object or objects.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present invention comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A computerized system, comprising:
a plurality of neuromuscular sensors configured to continuously record a plurality of neuromuscular signals from a user, wherein the plurality of neuromuscular sensors are arranged on one or more wearable devices; and
at least one computer processor programmed to:
determine, for each of the plurality of neuromuscular sensors at least one quality metric associated with the plurality of neuromuscular signals recorded by the neuromuscular sensor;
detect, in real-time, one or more artifacts in the recorded plurality of neuromuscular signals based on the at least one quality metric, wherein detecting one or more artifacts in the recorded plurality of neuromuscular signals comprises identifying a first neuromuscular sensor in which at least one quality metric deviates from a threshold value by more than a particular amount;
determine, based at least in part, on the detected one or more artifacts, a plurality of derived neuromuscular signals to mitigate the one or more artifacts, wherein determining the plurality of derived neuromuscular signals comprises:
selecting, based on the at least one quality metric, an artifact mitigation technique to mitigate the detected one or more artifacts; and
applying the artifact mitigation technique to the recorded plurality of neuromuscular signals to generate a plurality of derived neuromuscular signals in which the detected one or more artifacts have been at least partially removed; and
provide, as input to one or more trained statistical models, the plurality of derived neuromuscular signals.

2. The computerized system of claim 1, wherein selecting an artifact mitigation technique comprises determining whether to process the neuromuscular signals recorded by the first neuromuscular sensor to at least partially remove the one or more artifacts or whether to replace the neuromuscular signals recorded by the first neuromuscular sensor with neuromuscular signals recorded by at least one second neuromuscular sensor of the plurality of neuromuscular sensors, and wherein applying the artifact mitigation technique to the recorded plurality of neuromuscular signals comprises:
processing the neuromuscular signals recorded by the first neuromuscular sensor when it is determined to process the neuromuscular signals recorded by the first neuromuscular sensor; and
replacing the neuromuscular signals recorded by the first neuromuscular sensor with neuromuscular signals recorded by at least one second neuromuscular sensor of the plurality of neuromuscular sensors when it is determined to replace the neuromuscular signals recorded by the first neuromuscular sensor.

3. The computerized system of claim 2, wherein processing the neuromuscular signals comprises filtering the neuromuscular signals recorded by the first neuromuscular sensor to remove at least one external noise component.

4. The computerized system of claim 2, wherein processing the neuromuscular signals comprises substituting the neuromuscular signals with previously recorded neuromuscular signals from the first neuromuscular sensor.

5. The computerized system of claim 2, wherein determining whether to process the neuromuscular signals recorded by the first neuromuscular sensor or replace the neuromuscular signals recorded by the first neuromuscular sensor is based, at least in part, on a type of artifact associated with neuromuscular signals recorded by the first neuromuscular sensor.

6. The computerized system of claim 2, wherein determining whether to process the neuromuscular signals recorded by the first neuromuscular sensor or replace the neuromuscular signals recorded by the first neuromuscular sensor is based, at least in part, on a magnitude of the at least one quality metric.

7. The computerized system of claim 2, wherein detecting one or more artifacts in the recorded plurality of neuromuscular signals comprises identifying a first neuromuscular sensor in which the at least one quality metric deviates from a threshold value by more than a particular amount, and wherein determining, based at least in part, on the detected one or more artifacts, a plurality of derived neuromuscular signals comprises:
replacing the neuromuscular signals recorded by the first neuromuscular sensor with neuromuscular signals recorded by at least one second neuromuscular sensor of the plurality of neuromuscular sensors.

8. The computerized system of claim 7, wherein replacing the neuromuscular signals recorded by the first neuromuscular sensor comprises replacing the neuromuscular signals with an average of neuromuscular signals recorded by two or more other neuromuscular sensors of the plurality of neuromuscular sensors.

9. The computerized system of claim 8, wherein the two or more other neuromuscular sensors are arranged adjacent to the first neuromuscular sensor on the one or more wearable devices.

10. The computerized system of claim 1, wherein detecting one or more artifacts in the recorded plurality of neuromuscular signals comprises identifying a first neuromuscular sensor in which the at least one quality metric deviates from a threshold value by more than a particular amount, and wherein determining, based at least in part, on the detected one or more artifacts, a plurality of derived neuromuscular signals comprises:
processing the neuromuscular signals recorded by the first neuromuscular sensor.

11. The computerized system of claim 1, wherein the detected one or more artifacts are selected from the group consisting of noise artifacts, skin-contact artifacts, skin lift-off artifacts, power line frequency (e.g., 50 Hz, 60 Hz) artifacts, clipped signal artifacts, inactive sensor artifacts, microfriction artifacts, and data degeneration artifacts.

12. The computerized system of claim 1, wherein detecting the one or more artifacts in the recorded plurality of neuromuscular signals comprises analyzing the plurality of neuromuscular signals with a plurality of detector circuits, wherein each of the detector circuits is configured to detect a particular artifact.

13. The computerized system of claim 1, wherein the one or more trained statistical models include at least one trained statistical model trained using neuromuscular signals including the one or more artifacts.

14. The computerized system of claim 15, wherein the at least one trained statistical model is trained using derived neuromuscular signals that mitigate, at least in part, the one or more artifacts.

15. The computerized system of claim 1, wherein the at least one computer processor is further programmed to:
generate a musculoskeletal representation of a portion of a user based, at least in part, on an output of the one or more trained statistical models.

16. The computerized system of claim 15, wherein the musculoskeletal representation of the portion of the user is a musculoskeletal representation of a hand of the user.

17. The computerized system of claim 16, wherein the musculoskeletal representation of the hand includes position information and force information determined based, at least in part, on the output of the one or more trained statistical models.

18. The computerized system of claim 1, wherein the plurality of neuromuscular sensors comprise electromyography (EMG) sensors, mechanomyography (MMG) sensors, sonomyography (SMG) sensors, or a combination of two or more of EMG, MMG and SMG sensors.

19. A method of mitigating neuromuscular signal artifacts, the method comprising:
continuously recording a plurality of neuromuscular signals from a user using a plurality of neuromuscular sensors arranged on one or more wearable devices;
determining, for each of the plurality of neuromuscular sensors at least one quality metric associated with the plurality of neuromuscular signals recorded by the neuromuscular sensor;
detecting, in real-time, one or more artifacts in the recorded plurality of neuromuscular signals based on the at least one quality metric, wherein detecting one or more artifacts in the recorded plurality of neuromuscular signals comprises identifying a first neuromuscular sensor in which at least one quality metric deviates from a threshold value by more than a particular amount; and
determining, based at least in part, on the detected one or more artifacts, a plurality of derived neuromuscular signals to mitigate the one or more artifacts, wherein determining the plurality of derived neuromuscular signals comprises:
selecting, based on the at least one quality metric, an artifact mitigation technique to mitigate the detected one or more artifacts; and
applying the artifact mitigation technique to the recorded plurality of neuromuscular signals to generate a plurality of derived neuromuscular signals in which the detected one or more artifacts have been at least partially removed; and
providing as input to one or more trained statistical models, the plurality of derived neuromuscular signals.

20. A computer-readable medium encoded with a plurality of instructions that, when executed by at least one computer processor perform a method of:
continuously recording a plurality of neuromuscular signals from a user using a plurality of neuromuscular sensors arranged on one or more wearable devices;
determining, for each of the plurality of neuromuscular sensors at least one quality metric associated with the plurality of neuromuscular signals recorded by the neuromuscular sensor;
detecting, in real-time, one or more artifacts in the recorded plurality of neuromuscular signals based on the at least one quality metric, wherein detecting one or more artifacts in the recorded plurality of neuromuscular signals comprises identifying a first neuromuscular sensor in which at least one quality metric deviates from a threshold value by more than a particular amount; and
determining, based at least in part, on the detected one or more artifacts, a plurality of derived neuromuscular signals to mitigate the one or more artifacts, wherein determining the plurality of derived neuromuscular signals comprises:
selecting, based on the at least one quality metric, an artifact mitigation technique to mitigate the detected one or more artifacts; and
applying the artifact mitigation technique to the recorded plurality of neuromuscular signals to generate a plurality of derived neuromuscular signals in which the detected one or more artifacts have been at least partially removed; and
providing as input to one or more trained statistical models, the plurality of derived neuromuscular signals.
